(19) United States
(12) Patent Application Publication
(10) Pub. No.: US 2021/0128133 A1
(43) Pub. Date: May 6, 2021

(54) ORGAN RETRACTOR

(71) Applicants: Baxter International Inc., Deerfield, IL (US); Baxter Healthcare SA, Glattpark (Opfikon) (CH)

(72) Inventors: Sirisha Manam, Deerfield, IL (US); Caterina Rizzoni, Deerfield, IL (US); Stephen Truesdell, Deerfield, IL (US); Olivia Enneking, Deerfield, IL (US); Tony Siebel, Deerfield, IL (US); Ben Ko, Deerfield, IL (US); Elizabeth Dovec, Deerfield, IL (US)

(21) Appl. No.: 17/083,677

(22) Filed: Oct. 29, 2020

Related U.S. Application Data
(60) Provisional application No. 62/929,539, filed on Nov. 1, 2019.

(51) Int. Cl. A61B 17/02

(52) U.S. Cl. CPC A61B 17/0206; A61B 2017/0225

(57) ABSTRACT

An organ retractor includes a plurality of retractor jaws and a central housing. The central housing includes a threaded portion. The plurality of retractor jaws is hingedly connected to the central housing. The plurality of retractor jaws is disposed in an open position by a spring.

### PRIORITY CLAIM

This application claims priority to U.S. Provisional Patent Application No. 62/929,539, filed Nov. 1, 2019, entitled “ORGAN RETRACTOR,” the entire contents of which are hereby incorporated in its entirety herein, including all tables, figures, and claims.

### BACKGROUND

Surgical procedures, such as gastrointestinal surgical procedures, often require access to unique locations within the body. For example, in a minimally invasive gastric-bypass procedure, the surgical devices are passed through a trocar and are manipulated (manually or remotely) to access the stomach. While clear access to the stomach is necessary to properly perform this procedure, unfortunately other organs and anatomical features may prevent, or at least make difficult, access to the stomach. For example, the liver's position in the body typically means that it is resting “on top” of the stomach when the patient is lying down during surgery. In this particular example, to access the stomach the liver must be retracted or held away from the stomach.

A typical organ retractor used in gastrointestinal surgical procedures is a Nathanson liver retractor. The Nathanson liver retractor requires a physical mount, such as a mount that is affixed to the patient's bed or a surgical stand, and a number of support arms for directional adjustment of a retractor. The retractor further includes a hook-like arm, which extends into the patient's body. This hook-like arm is used to retract the liver away from the surgical environment. However, this retraction device is disadvantageous for a number of reasons. The Nathanson liver retractor requires a number of components disposed outside the patient's body, such as the physical mount and the support arms. Each of these components must be sterilized prior to use. Further, each of these components requires setup and proper orientation in the operating room. The hook-like arm is typically a metal hook, such as a 5 mm hook. To insert this hook-like arm into the patient, the surgeon must create a 5 mm incision on the patient's abdomen; the surgeon must also stitch up this incision after surgery. Additionally, the hook-like arm puts unnecessary localized pressure onto the liver, during retraction. In some circumstances, this localized pressure is so severe as to cause trauma, such as hematoma, to the liver. Further, it is often difficult to remove the hook-like arm from the body, once the procedure is completed, given the hook-like arm's geometry.

Improved systems and methods for organ retraction are therefore needed.

### SUMMARY

To improve the retraction of liver, and other related organs, in the surgical environment, new retraction systems and methods are provided herein.

In light of the disclosure herein, and without limiting the scope of the invention in any way, in a first aspect of the present disclosure, which may be combined with any other aspect listed herein unless specified otherwise, an organ retractor includes a plurality of retractor jaws and a central housing. The central housing includes a threaded portion. The plurality of retractor jaws is hingedly connected to the central housing. The plurality of retractor jaws is disposed in an open position by a spring.

In a second aspect of the present disclosure, which may be combined with any other aspect listed herein unless specified otherwise, the plurality of retractor jaws includes a non-woven mesh coupled to each of the plurality of retractor jaws and disposed between the plurality of retractor jaws.

In a third aspect of the present disclosure, which may be combined with any other aspect listed herein unless specified otherwise, the non-woven mesh is sized and shaped to retract a liver.

In a fourth aspect of the present disclosure, which may be combined with any other aspect listed herein unless specified otherwise, the plurality of retractor jaws includes two retractor jaws.

In a fifth aspect of the present disclosure, which may be combined with any other aspect listed herein unless specified otherwise, the organ retractor further includes a threaded obturator, configured to engage with the threaded portion of the central housing.

In a sixth aspect of the present disclosure, which may be combined with any other aspect listed herein unless specified otherwise, responsive to engaging the threaded obturator with the threaded portion of the central housing, the organ retractor is capable of being manipulated via the threaded obturator.

In a seventh aspect of the present disclosure, which may be combined with any other aspect listed herein unless specified otherwise, manipulation includes lifting the threaded obturator, such that the organ retractor is lifted to support an organ.

In an eighth aspect of the present disclosure, which may be combined with any other aspect listed herein unless specified otherwise, responsive to lifting the threaded obturator, the threaded obturator is clamped at an exterior of a patient, such that the threaded obturator is prevented from sliding within an incision on the patient.

In a ninth aspect of the present disclosure, which may be combined with any other aspect listed herein unless specified otherwise, the central housing includes an end piece, configured to be manipulated by a surgical grasping tool.

In a tenth aspect of the present disclosure, which may be combined with any other aspect listed herein unless specified otherwise, the plurality of retractor jaws and central housing are configured to be inserted, into a patient, via a port.

In an eleventh aspect of the present disclosure, which may be combined with any other aspect listed herein unless specified otherwise, the plurality of retractor jaws are inserted into the patient in a closed configuration.

In a twelfth aspect of the present disclosure, which may be combined with any other aspect listed herein unless specified otherwise, responsive to exiting the port and entering the patient, the plurality of retractor jaws are disposed into the open position via the spring.

In a thirteenth aspect of the present disclosure, which may be combined with any other aspect listed herein unless specified otherwise, an organ retractor includes two retractor jaws, hingedly coupled to one another, and a non-woven mesh. The non-woven mesh is coupled to each of the retractor jaws and disposed between the retractor jaws. The retractor jaws are disposed in an open position by a spring.

In a fourteenth aspect of the present disclosure, which may be combined with any other aspect listed herein unless specified otherwise, the non-woven mesh is sized and shaped to retract a liver.

In a fifteenth aspect of the present disclosure, which may be combined with any other aspect listed herein unless specified otherwise, the organ retractor further includes a central housing including a threaded portion, wherein the retractor jaws are hingedly connected to the central housing.

In a sixteenth aspect of the present disclosure, which may be combined with any other aspect listed herein unless specified otherwise, the organ retractor further includes a threaded obturator, configured to engage with the threaded portion of the central housing.

In a seventeenth aspect of the present disclosure, which may be combined with any other aspect listed herein unless specified otherwise, responsive to engaging the threaded obturator with the threaded portion of the central housing, the organ retractor is capable of being manipulated via the threaded obturator.

In an eighteenth aspect of the present disclosure, which may be combined with any other aspect listed herein unless specified otherwise, manipulation includes lifting the threaded obturator, such that the organ retractor is lifted to support an organ.

In a nineteenth aspect of the present disclosure, which may be combined with any other aspect listed herein unless specified otherwise, responsive to lifting the threaded obturator, the threaded obturator is clamped at an exterior of a patient, such that the threaded obturator is prevented from sliding within an incision on the patient.

In a twentieth aspect of the present disclosure, which may be combined with any other aspect listed herein unless specified otherwise, the central housing includes an end piece, configured to be manipulated by a surgical grasping tool.

In a twenty-first aspect of the present disclosure, any of the structure, functionality, and alternatives discussed in connection with any of FIGS. 1 to 14 may be combined with any of the structure, functionality, and alternatives discussed in connection with any other one or more of FIGS. 1 to 14.

In light of the disclosure and aspects set forth herein, it is accordingly an advantage of the present disclosure to provide an organ retractor with simplified structure for support, improving ease of use and reducing the number of components needed.

It is another advantage of the present disclosure to provide an organ retractor with a reduced footprint, so as to utilize existing trocars and avoid additional large incisions.

It is a further advantage of the present disclosure to provide an organ retractor that retracts organs over a distributed surface, so as to reduce risk of trauma.

It is yet another advantage of the present disclosure to provide an organ retractor that is easy to manipulate within the patient's body cavity and is, likewise, easy to remove from the patient's body cavity.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE FIGURES

Understanding that figures depict only typical embodiments of the invention and are not to be considered to be limiting the scope of the present disclosure, the present disclosure is described and explained with additional specificity and detail through the use of the accompanying figures. The figures are listed below.

FIG. 1A to 1C illustrate top and side views of an organ retractor and threaded obturator, according to an example embodiment of the present disclosure.

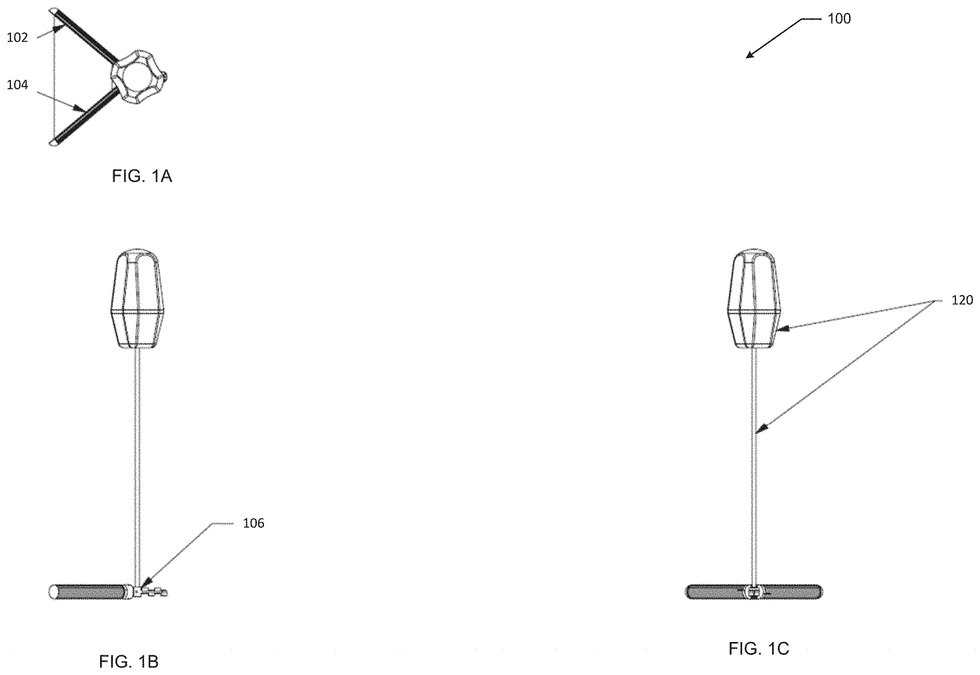

FIG. 2 illustrates an exploded perspective view of an organ retractor and threaded obturator, according to an example embodiment of the present disclosure.

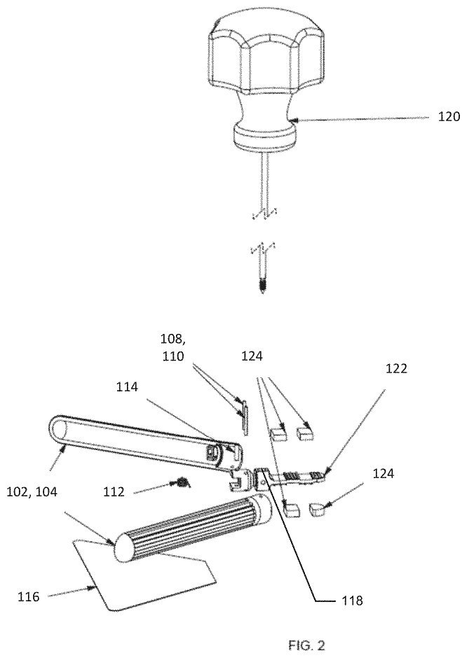

FIG. 3 illustrates a side view of a threaded obturator, according to an example embodiment of the present disclosure.

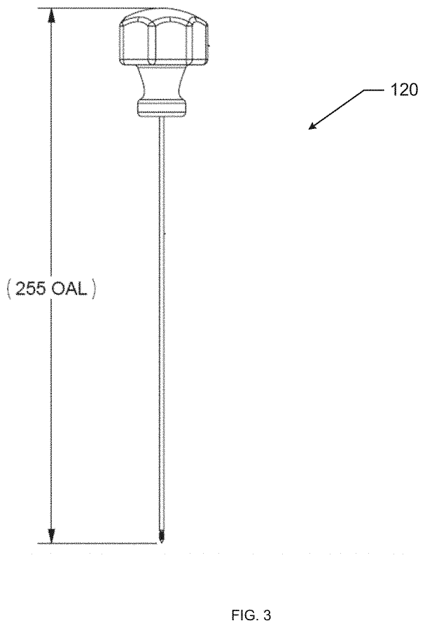

FIG. 4 illustrates a side view of a shaft of a threaded obturator, according to an example embodiment of the present disclosure.

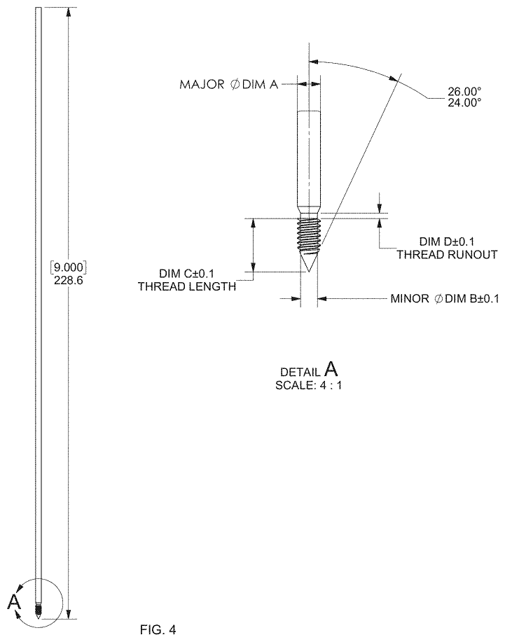

FIG. 5A to 5C illustrate top, side, and sectioned views of a handle of a threaded obturator, according to an example embodiment of the present disclosure.

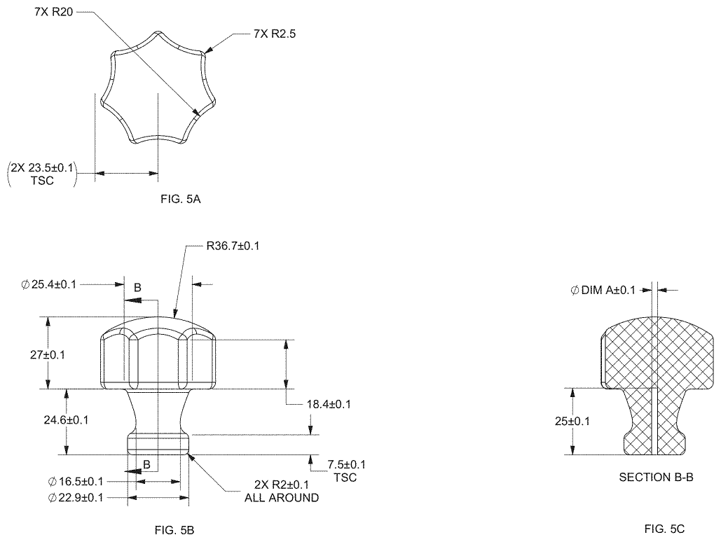

FIG. 6A to 6C illustrate top, side, and sectioned views of a handle of a threaded obturator, according to an example embodiment of the present disclosure.

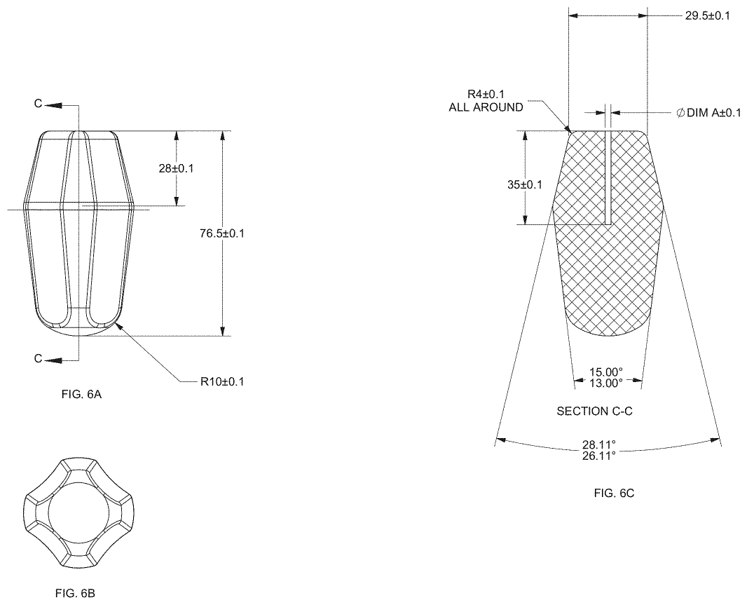

FIG. 7A to 7C illustrate front, sectioned, and bottom views of an end piece, according to an example embodiment of the present disclosure.

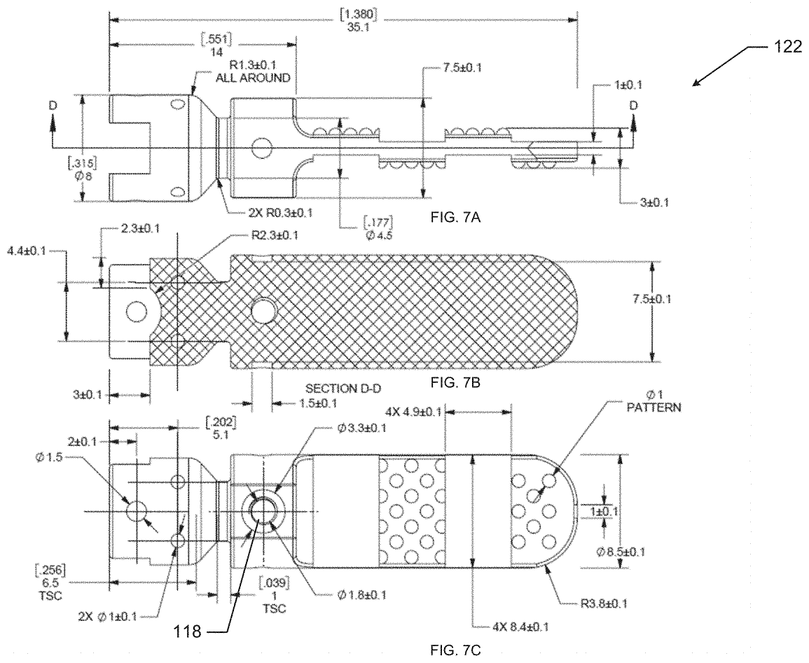

FIG. 8A to 8B illustrate front and bottom views of an end piece, according to an example embodiment of the present disclosure.

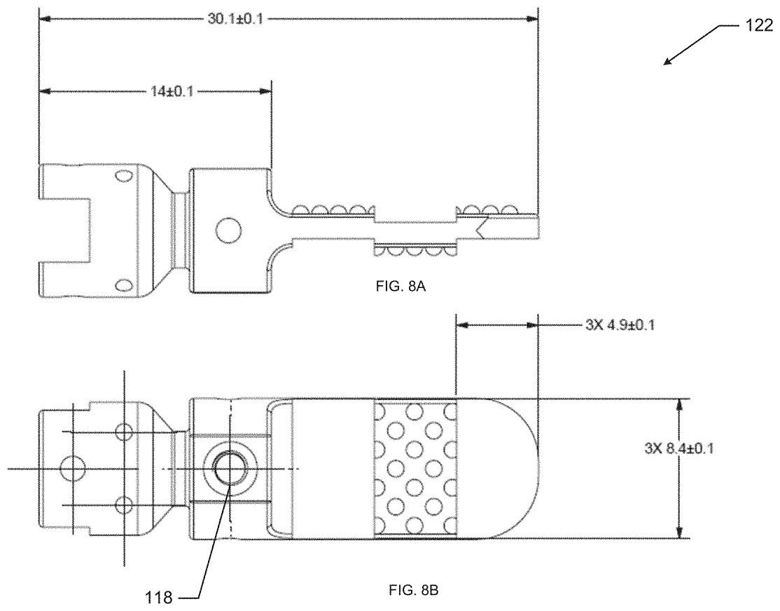

FIG. 9A to 9D illustrate top, front, bottom, and sectioned views of an organ retractor, according to an example embodiment of the present disclosure.

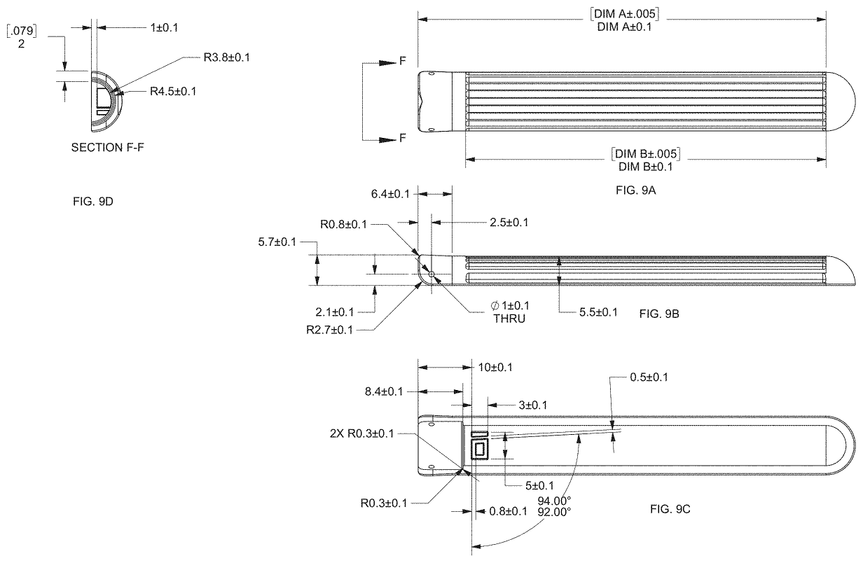

FIG. 10 illustrates a front view of a center spring pin, according to an example embodiment of the present disclosure.

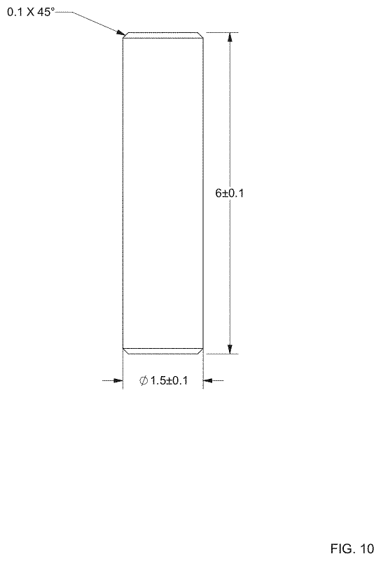

FIG. 11 illustrates a front view of a hinge pin, according to an example embodiment of the present disclosure.

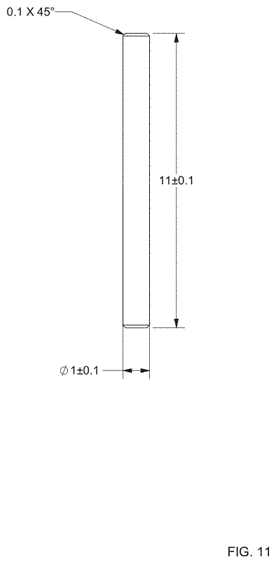

FIG. 12A to 12B illustrate top and front views of a torsion spring, according to an example embodiment of the present disclosure.

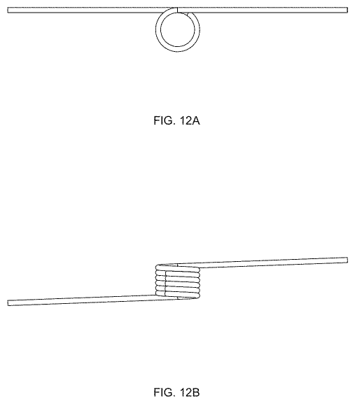

FIG. 13A to 13B illustrate top and front views of foam pieces, according to an example embodiment of the present disclosure.

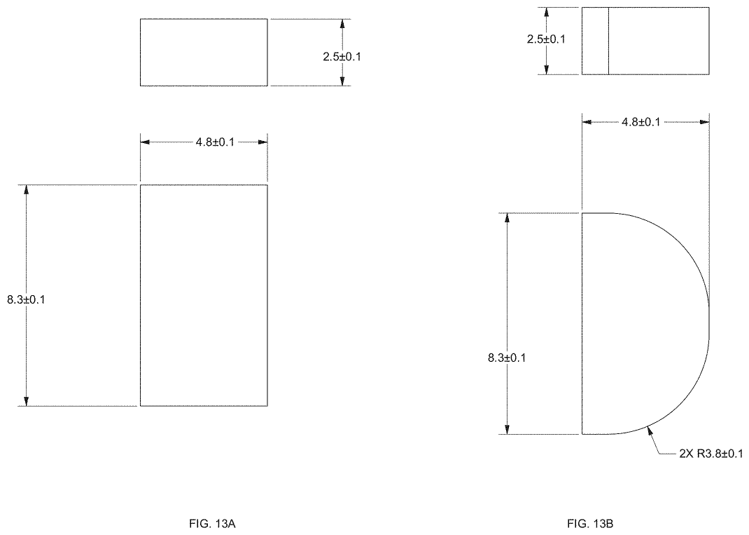

FIG. 14 illustrates top and front views of a non-woven mesh, according to an example embodiment of the present disclosure.

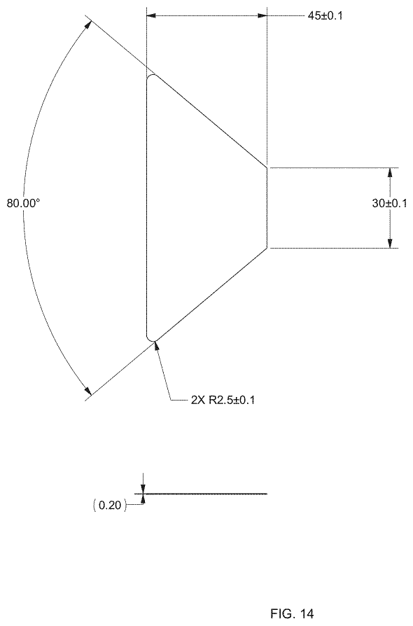

### DETAILED DESCRIPTION

Certain embodiments described herein relate generally to the field of organ retraction. More particularly, some embodiments described herein relate to the retraction of organs, such as a liver, to improve access to the surgical environment.

Referring now to the drawings, an organ retractor 100 is shown in an open configuration. The organ retractor includes at least two retractor jaws, such as a first retractor jaw 102 and a second retractor jaw 104. Each of the first retractor jaw 102 and the second retractor jaw 104 is hingedly connected to a central housing 106 of the organ retractor 100. Namely, the first retractor jaw 102 and the second retractor jaw 104 are hingedly connected to the central housing 106 via a plurality of j aw hinge pins 108, 110. For example, each of the first retractor jaw 102 and the second retractor jaw 104 pivot about one of the jaw hinge pins 108, 110.

The central housing 106 also includes a torsion spring 112 connected to the central housing 106 via a center spring pin 114. For example, the center spring pin 114 passes through a central loop of the torsion spring 112. The torsion spring 112 engages with each of the first retractor jaw 102 and the second retractor jaw 104. For example, an offshoot from the torsion spring 112 engages with the first retractor jaw 102; likewise, an offshoot from the torsion spring 112 engages with the second retractor jaw 104. Generally, the torsion spring 112 urges the first retractor jaw 102 and the second retractor jaw 104 away from each other, such that the organ retractor 100 is generally disposed in the open configuration by the torsion spring 112. In the open configuration, the first retractor jaw 102 and the second retractor jaw 104 generally form a v-shape.

The organ retractor 100 further includes a mesh or fabric, such as a non-woven mesh 116. In an example, the non-woven mesh 116 is affixed to each of the first retractor jaw 102 and the second retractor jaw 104. When the organ retractor 100 is in the open configuration, the non-woven mesh 116 is generally a taught surface between the first retractor jaw 102 and the second retractor jaw 104. In an embodiment, the non-woven mesh 116 is sized and shaped to engage with a portion of a liver. It should be noted that the non-woven mesh 116 is flexible; namely, when the organ retractor 100 is in a closed configuration (prior to insertion, for example), the non-woven mesh 116 is collapsible.

The central housing 106 of the organ retractor 100 includes a threaded portion 118, such as a threaded hole or aperture. The threaded portion 118 is disposed on the organ retractor 100 proximate to the center spring pin 114. In an embodiment, the threaded portion 118 is disposed along an axis that is perpendicular to a plane defined by the non-woven mesh 116 when the organ retractor 100 is in the open configuration.

The threaded portion 118 is generally configured to engage with a threaded obturator 120. For example, a surgeon can rotate the threaded obturator 120 to engage with the organ retractor 100, and subsequently manipulate the organ retractor 100 via the threaded obturator 120, as described in greater detail herein.

The central housing 106 of the organ retractor 100 may further include an end piece 122. In an embodiment, the end piece 122 may extend distally from the threaded portion 118. The end piece 122 may be generally rectangular in shape, although other shapes are contemplated. The end piece 122 may include a plurality of foam pieces, ridges, or other surface features 124. For example, a surgeon may grab the organ retractor 100 at the end piece 122, and subsequently remove the organ retractor 100, via a surgical grasping instrument, as described in greater detail herein.

In light of the disclosure above regarding the structural components of the organ retractor 100, it is helpful to explain how the organ retractor 100 is used in the surgical environment.

In typical gastrointestinal surgeries, a 12 mm trocar is implanted onto the patient's abdomen, to provide the surgeon with a window of access into the patient's body cavity. The surgeon will manually collapse the first retractor jaw 102 and the second retractor jaw 104 together (against the spring force of the torsion spring 112), and subsequently insert the organ retractor 100 through the 12 mm trocar. In a preferred embodiment, the organ retractor 100 is inserted such that the end piece 122 is the last component to enter the 12 mm trocar. Once the organ retractor 100 passes through the 12 mm trocar, and enters the patient's body cavity, the organ retractor 100 will expand to the open configuration. Namely, each of the first retractor jaw 102 and second retractor jaw 104 are urged away from each other by the torsion spring 112.

The surgeon then passes the threaded obturator 120 through a small incision on the patient. In an embodiment, the incision is a 2 mm incision. Via cameras or other optical techniques, the surgeon locates the threaded portion 118 of the organ retractor 100, and subsequently engages the threaded obturator 120 with this threaded portion 118 by twisting the handle of the threaded obturator 120. Once the threaded obturator 120 is engaged with the organ retractor 100, the surgeon may position the organ retractor 100 against or underneath an organ, such as the liver. The surgeon then “lifts” or “pulls” on the threaded obturator 120, such that the non-woven mesh 116 retracts the organ. For example, the surgeon positions the organ retractor 100 under the liver and lifts up, such that the liver is moved out of the way of the stomach.

The threaded obturator 120 is then clamped in place outside of the patient's body cavity. For example, a clip or other mechanical component is fastened to the shaft of the threaded obturator 120 near the exterior surface of the patient's skin. This may advantageously ensure that the shaft of the threaded obturator 120 remains fixed and does not slide. Thus, the organ retractor 100 remains fixed.

Upon completion of the surgical procedure, once surgical access is no longer necessary, the clip or other mechanical component is removed from the shaft of the threaded obturator 120. The surgeon then disengages the threaded obturator 120 from the organ retractor 100 by un-twisting the handle of the threaded obturator 120. The threaded obturator 120 is removed from the surgical environment (via the small incision). The surgeon then grasps the organ retractor 100 via a surgical grasping tool. Specifically, for example, the surgeon passes a surgical grasping tool through the 12 mm trocar; the surgeon grasps the end piece 122 of the organ retractor 100 with the surgical grasping tool. Once the organ retractor 100 is grasped, the surgeon can “pull” the organ retractor 100 out of the patient's body cavity through the 12 mm trocar. Due to the hinged orientation of the first retractor jaw 102 and the second retractor jaw 104, the 12 mm trocar will “close” each of the first retractor jaw 102 and the second retractor jaw 104 as the organ retractor 100 is pulled through the 12 mm trocar by the end piece 122.

This organ retractor 100 and related procedures disclosed herein advantageously eliminate additional external support structures, thus improving setup time and simplifying the retraction procedure. The organ retractor 100 and related procedures utilize existing trocars and eliminate the need for a larger 5 mm incision, thus reducing the total number of incisions and improving patient outcomes. The organ retractor 100 and related procedures retract organs over a distributed surface, thus reducing risk of patient trauma such as hematoma. The organ retractor and related procedures are generally easier to manipulate, affix, and remove.

As used in this specification, including the claims, the term “and/or” is a conjunction that is either inclusive or exclusive. Accordingly, the term “and/or” either signifies the presence of two or more things in a group or signifies that one selection may be made from a group of alternatives.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. Note that elements recited in means-plus-function format are intended to be construed in accordance with 35 U.S.C. § 112 ¶ 6. The scope of the invention is therefore defined by the following claims.

The invention is claimed as follows:

1. An organ retractor comprising:
a plurality of retractor jaws; and
a central housing including a threaded portion,
wherein the plurality of retractor jaws is hingedly connected to the central housing, and
wherein the plurality of retractor jaws is disposed in an open position by a spring.

2. The organ retractor of claim 1, wherein the plurality of retractor jaws includes a non-woven mesh coupled to each of the plurality of retractor jaws and disposed between the plurality of retractor jaws.

3. The organ retractor of claim 2, wherein the non-woven mesh is sized and shaped to retract a liver.

4. The organ retractor of claim 1, wherein the plurality of retractor jaws includes two retractor jaws.

5. The organ retractor of claim 1, further comprising a threaded obturator, configured to engage with the threaded portion of the central housing.

6. The organ retractor of claim 5, wherein, responsive to engaging the threaded obturator with the threaded portion of the central housing, the organ retractor is capable of being manipulated via the threaded obturator.

7. The organ retractor of claim 6, wherein manipulation includes lifting the threaded obturator, such that the organ retractor is lifted to support an organ.

8. The organ retractor of claim 7, wherein, responsive to lifting the threaded obturator, the threaded obturator is clamped at an exterior of a patient, such that the threaded obturator is prevented from sliding within an incision on the patient.

9. The organ retractor of claim 1, wherein the central housing includes an end piece, configured to be manipulated by a surgical grasping tool.

10. The organ retractor of claim 1, wherein the plurality of retractor jaws and central housing are configured to be inserted, into a patient, via a port.

11. The organ retractor of claim 10, wherein the plurality of retractor jaws are inserted into the patient in a closed configuration.

12. The organ retractor of claim 11, wherein, responsive to exiting the port and entering the patient, the plurality of retractor jaws are disposed into the open position via the spring.

13. An organ retractor comprising:
two retractor jaws, hingedly coupled to one another; and
a non-woven mesh coupled to each of the retractor jaws and disposed between the retractor jaws,
wherein the retractor jaws are disposed in an open position by a spring.

14. The organ retractor of claim 13, wherein the non-woven mesh is sized and shaped to retract a liver.

15. The organ retractor of claim 13, further comprising a central housing including a threaded portion, wherein the retractor jaws are hingedly connected to the central housing.

16. The organ retractor of claim 15, further comprising a threaded obturator, configured to engage with the threaded portion of the central housing.

17. The organ retractor of claim 16, wherein, responsive to engaging the threaded obturator with the threaded portion of the central housing, the organ retractor is capable of being manipulated via the threaded obturator.

18. The organ retractor of claim 17, wherein manipulation includes lifting the threaded obturator, such that the organ retractor is lifted to support an organ.

19. The organ retractor of claim 18, wherein, responsive to lifting the threaded obturator, the threaded obturator is clamped at an exterior of a patient, such that the threaded obturator is prevented from sliding within an incision on the patient.

20. The organ retractor of claim 15, wherein the central housing includes an end piece, configured to be manipulated by a surgical grasping tool.
